(19) United States
(12) Patent Application Publication
(10) Pub. No.: US 2011/0213318 A1
(43) Pub. Date: Sep. 1, 2011

(54) Intermittent catheter having a stiff distal section and method of manufacturing such

(75) Inventor: Lars Olav Schertiger, Fredensborg (DK)

(73) Assignee: COLOPLAST A/S, Humlebaek (DK)

(21) Appl. No.: 13/128,411

(22) PCT Filed: Nov. 13, 2009
(86) PCT No.: PCT/DK09/50302

§ 371 Date: May 10, 2011

(30) Foreign Application Priority Data
Nov. 14, 2008 (DK) PA 2008 01595

(51) Int. Cl. A61M 25/00; B29C 65/54

(52) U.S. Cl. 604/264; 156/294

(57) ABSTRACT

In the following there is disclosed a catheter for voiding a cavity in the human body. The catheter comprises a first tubular catheter part extending longitudinally between a proximal end provided with at least one inlet, and a distal end provided with at least one outlet and a second tubular catheter part extending longitudinally between a proximal end provided with one opening and a distal end provided with a second opening. The catheter further comprises that the first tubular catheter part is longer than the second tubular catheter part; that the second tubular catheter part is arranged on the first tubular catheter at the distal end; and that the first tubular catheter part and the second tubular catheter part are permanently displaceable fixed relative to each other. This provides a catheter which has distal end which is more stable than conventional intermittent catheters and thus is easier to insert with a reduced risk that the catheter bends or kinks during catherisation.

### TECHNICAL FIELD

The present invention relates to a catheter and a method of producing such, which is easy to insert and manufacture.

### BACKGROUND

Intermittent catherisation is often used in order to void the bladder for people who are not capable of voluntarily do so. Today many types of catheters are available, many suitable for different urinary channel sizes and urethra lengths. In particular the urethra of men is longer than that of women and thus longer catheters are needed for men in order to successfully void the bladder.

At the same time a certain amount of flexibility of the catheter is necessary for it to follow the curvature of the urethra, for example when passing the prostate.

However, long and flexible catheters are difficult to handle as the catheter tend to bend or kink unintentionally during insertion, which complicates the catherisation process. In particular as a large number of intermittent catheter user already might have a handicap which impairs their mobility and/or motor skills such unintentional bend or kink may be very frustrating. In order to stabilize the catheter they might grab hold of the catheter body thereby contaminating the part which is inserted into the urethra and thus increasing the chance the chance of tract infection.

In order to address this problem a lot of catheter sets are incorporating a guide which may be slid along the catheter body without contaminating and while facilitating insertion.

Although these function very well, they do contain that extra guide component which has to be handled simultaneously while inserting and which accidentally can be dropped and thus out of reach for a person who have decreased mobility.

Thus, there exists a need for a intermittent catheter which is easy to insert, having a reduced risk of unintentionally bending or kinking, without the use of a guiding means.

### SUMMARY OF THE INVENTION

In the following there is disclosed a catheter for voiding a cavity in the human body, comprising a first tubular catheter part extending longitudinally between a proximal end provided with at least one inlet, and a distal end provided with at least one outlet and a second tubular catheter part extending longitudinally between a proximal end provided with one opening and a distal end provided with a second opening. The catheter further comprises that the first tubular catheter part is longer than the second tubular catheter part; that the second tubular catheter part is arranged on the first tubular catheter at the distal end; and that the first tubular catheter part and the second tubular catheter part are permanently displaceable fixed relative to each other.

This provides a catheter which has distal end which is more stable than conventional intermittent catheters and thus is easier to insert with a reduced risk that the catheter bends or kinks during catherisation.

In one embodiment the second tubular catheter part has a higher rigidity than the first tubular catheter part. This provides an even more stable distal section of the catheter and thus makes it even easier to insert the catheter.

In one embodiment the first catheter part has an outer radius which is larger than the inner radius of the second catheter part.

Typically, a connector is attached to the catheter so that it is possible to connect the catheter to a urine receptacle via a hose. Thus in one embodiment, a connector is attached to the distal end of the first tubular catheter part.

In another embodiment the distal end of the second tubular catheter part may additionally or alternatively be attached to the connector.

In one embodiment the connector could be integrally formed with the second tubular catheter part, for example by injection molding.

In another embodiment the two catheter parts overlap each other, i.e. the distal end of the first tubular catheter part is attached to the second tubular catheter part between the distal and proximal end of the second tubular catheter part.

The two catheter part can be attached to each other in many different ways. For example can the first tubular catheter part and the second tubular catheter part be displaceable fixed relative to each other by means of an adhesive or by means of a weld.

A method of manufacturing a catheter as described is also disclosed herein. The catheter comprises a proximal section having at least one inlet arranged at the proximal end for insertion into the bladder and a distal section having at least one outlet provided at the distal end allowing urine exiting the bladder to be evacuated. The catheter further comprises a first tubular catheter part extending longitudinally between a proximal end provided with at least one inlet, and a distal end provided with at least one outlet; a second tubular catheter part extending longitudinally between a proximal end provided with one opening and a distal end provided with a second opening; and that the first tubular catheter part is longer than the second tubular catheter part. The method of manufacturing the catheter comprises that the second tubular catheter part is guided coaxially onto the first tubular catheter part by introducing the first tubular catheter part into one of the openings of the second tubular catheter part; that the second tubular catheter part is arranged at the distal end of the first tubular catheter part, and that the first tubular catheter part and the second tubular catheter part are permanently displaceable fixed relative to each other.

This provides a method which uses simple steps to provide a catheter as described herein. By using intermediate products which are simple to produce, i.e. the first and second catheter part, the method is fast and cheap while providing a reliable catheter which has a stable distal end which provides a catheter which is easy to insert and guide.

In one embodiment in order to fix the catheter parts relative to each other the second tubular catheter part is attached to the first tubular catheter part by an adhesive. Alternatively or additionally it may be attached by welding.

Many other means of permanently fixing the two catheter parts to each other can be used. For example using hot-melt or heat shrink sleeves can be used overlapping parts in order to hold them together, e.g. overlap the second tubular catheter part and the connector in cases where the connector is attached to the first tubular catheter part.

Other means to fix the two parts may be to provide matching threads on the two parts so they may be screwed together.

Alternatively other additionally the part can be riveted together. This can for example be done by using ultrasound punching, where some of the material of the second tubular catheter part is punched partly into the first tubular catheter, locking the two pieces together.

In order to obtain increased stability the distal end of the first tubular catheter part should be axially aligned with the distal end of the second tubular catheter part, or at least as close as possible. This reduces the risk that the catheter bends or kinks at the distal end.

In one embodiment the distal end of the first tubular catheter part is arranged between the distal and proximal end of the second tubular catheter part. This allows the production of different length catheter by using the same tubular catheter parts by fixing the two catheter parts in different axial displacements.

### BRIEF DESCRIPTION OF THE DRAWING

FIG. 1 shows in section a catheter as disclosed herein.

### DETAILED DESCRIPTION

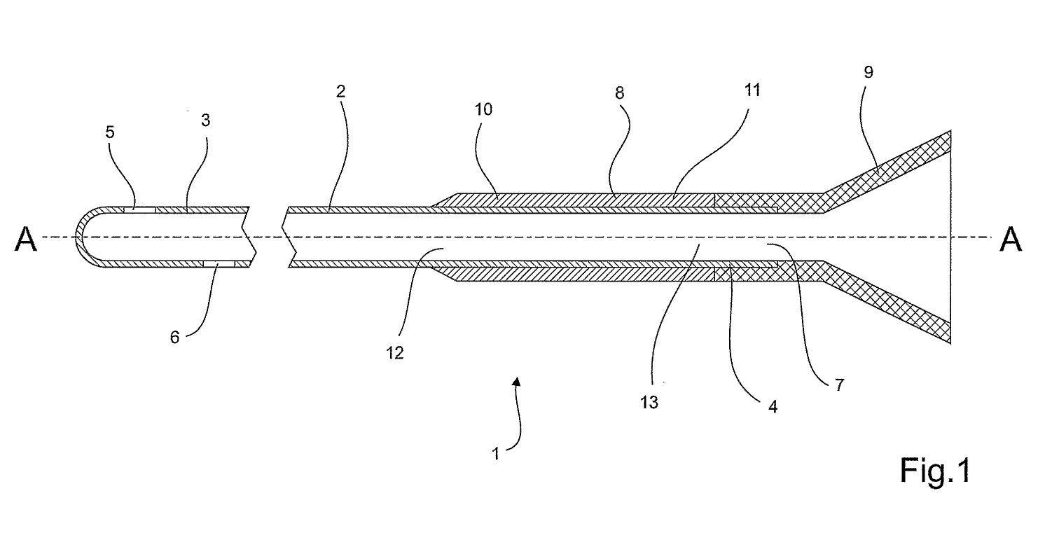

One embodiment of a catheter 1 for voiding a cavity in the human body (not shown) as disclosed herein is shown in FIG. 1 The catheter is shown in section along its longitudinal axis A-A.

The catheter 1 comprises a first tubular catheter part 2, a second tubular catheter part 8, and a connector 9.

The first tubular catheter part 2 extends longitudinally along axis A-A between a proximal end 3 and a distal end 4. At the proximal end there is provided two so-called eyelets 5,6 which functions as inlets; and in the distal end there is provided one outlet 7. At the distal end and in communication with the outlet the connector 9 is attached. This allows the first catheter part to be attached to a hose which may guide urine to a urine bag or other type of receptacle.

For example, as a first tubular catheter part the Speedicath intermittent catheter sold by Coloplast A/S can be used.

The second tubular catheter part 8 extends longitudinally along axis A-A between a proximal end 10 and a distal end 11. The proximal end is provided with one opening 12 and the distal end is provided with a second opening 13.

The second tubular catheter part 8 has an inner tubular radius which allows it to be arranged on the first tubular catheter part 2 by inserting the first tubular catheter part through the first 12 or the second opening 13 of the second tubular catheter part. The second tubular catheter part 8 is shorter than the first tubular catheter part. In the present embodiment the first tubular catheter part is approximately four times longer than the second tubular catheter part.

Even though the first tubular catheter part has a slightly larger radius than the second tubular catheter part it is possible to guide it through the first and/or second opening due to the compressible properties of the first tubular catheter. Typically this is due to the thin walls of the first catheter part and the material which for example could be polyurethane. Even more so, this provided a frictional fit and reduces the risk of gaps between the two catheter parts wherein mucous might get caught.

In other embodiments, not shown herein, the first tubular catheter part may have a slightly smaller radius. This might be necessary due to production tolerances. However, it has shown that a gap between the inner surface of the second tubular catheter part and the outer surface of the first tubular catheter part of around 0.3 mm can be used without risk of tearing mucosa.

In order to facilitate insertion, and reduce the risk that the mucosa is torn, the proximal end of the second tubular catheter part can be provided with a tapered shape inwards toward the surface of the first tubular catheter part.

In one example, where the first tubular catheter part is a Speedicath intermittent catheter as described above, the proximal end of the first tubular catheter part is inserted through the second opening 13. It is then guided all the way through until the connector 9 prevents any further movement. The second tubular catheter part is then attached to the connector in order to prevent any undesired displacement. Alternatively or additionally the two catheter parts could also be attached to each other.

Thus, a catheter as described has a higher stiffness at its distal end which allows it to be easier inserted and guided into the urethra. In particular for men who has a longer urethra this is could be a problem as a relative long catheter is more difficult to handle and have a tendency to more easily bend/kink unintentionally during insertion.

Such a catheter is also easy to manufacture as no complicated manufacture steps are needed. For example as mentioned above the first tubular catheter part, in the form of the Speedicath intermittent catheter, is already provided by known well-established production methods and the second tubular catheter may also be manufactured by well-known production methods such as injection-molding.

### REFERENCE NUMBERS

1. catheter

2. first tubular catheter part

3. proximal end of the first tubular part

4. distal end of the first tubular part

5, first eyelet

6. second eyelet

7. outlet

8. second tubular catheter part

9. connector

10. proximal end of the second tubular part

11. distal end of the second tubular catheter

12. one opening

13. second opening

1. A method of manufacturing a catheter insertable into a bladder for evacuation of urine from the bladder the method comprising:
providing a first tubular catheter part extending longitudinally between a proximal end provided with at least one inlet, and a distal end provided with at least one outlet,
providing a second tubular catheter part extending longitudinally between a proximal end provided with one opening and a distal end provided with a second opening,
the first tubular catheter part is longer than the second tubular catheter part,
guiding the second tubular catheter part coaxially onto the first tubular catheter part by introducing the first tubular catheter part into one of the openings of the second tubular catheter part,
arranging the second tubular catheter part distal the distal end of the first tubular catheter part, and
fixing the first tubular catheter part and the second tubular catheter part into permanent engagement relative to each other.

2. A method of manufacturing a catheter according to claim 1, wherein the second tubular catheter part is attached to the first tubular catheter part by an adhesive.

3. A method of manufacturing a catheter according to claim 1, wherein the second tubular catheter part is attached to the first tubular catheter part by welding.

4. A method according to claim 1, wherein the distal end of the first tubular catheter part is axially aligned with the distal end of the second tubular catheter part.

5. A method according to claim 1, wherein the distal end of the first tubular catheter part is arranged between the distal and proximal end of the second tubular catheter part.

6. A catheter for voiding a cavity in the human body, comprising
a first tubular catheter part extending longitudinally between a proximal end provided with at least one inlet, and a distal end provided with at least one outlet,
a second tubular catheter part extending longitudinally between a proximal end provided with one opening and a distal end provided with a second opening, and
that the first tubular catheter part is longer than the second tubular catheter part,
that the second tubular catheter part is arranged on the first tubular catheter at the distal end, and
that the first tubular catheter part and the second tubular catheter part move relative to each other into a permanently fixed relationship.

7. A catheter according to claim 6, wherein the second tubular catheter part has a higher stiffness than the first tubular catheter part.

8. A catheter according to claim 6, wherein the first catheter part has an outer radius that is larger than the inner radius of the second catheter part.

9. A catheter according to claim 6, wherein a connector is attached to the distal end of the first tubular catheter part.

10. A catheter according to claim 9, wherein the distal end of the second tubular catheter part is attached to the connector.

11. A catheter according to claim 6, wherein a connector is attached to the distal end of the second tubular catheter part.

12. A catheter according to claim 11, wherein the distal end of the first tubular catheter part is attached to the second tubular catheter part between the distal and proximal end of the second tubular catheter part.

13. A catheter according to claim 11, wherein the connector is integrally formed with the second tubular catheter part.

14. A catheter according to claim 6, wherein the first tubular catheter part and the second tubular catheter part are fixed relative to each other by an adhesive.

15. A catheter according to claim 6, wherein the first tubular catheter part and the second tubular catheter part are fixed relative to each other by a weld.
